(19) United States
(12) Patent Application Publication
(10) Pub. No.: US 2004/0221843 A1
(43) Pub. Date: Nov. 11, 2004

(54) Evaporator, especially a respiratory humidifier, storage tank and casing therefor

(76) Inventor: Martin Baecke, Dessau (DE)

Correspondence Address: LAW OFFICES OF CLEMENT CHENG, 17220 NEWHOPE STREET #127, FOUNTAIN VALLEY, CA, 92708, US

(21) Appl. No.: 10/826,664

(22) Filed: Apr. 17, 2004

Related U.S. Application Data
(63) Continuation of application No. PCT/DE02/03890, filed on Oct. 15, 2002.

(30) Foreign Application Priority Data
Oct. 18, 2001 (DE) 101 51 397.6

(51) Int. Cl. A61M 15/00; A61M 16/10

(52) U.S. Cl. 128/203.16; 128/203.12

(57) ABSTRACT

The present application relates to an evaporator, especially a respiratory humidifier, comprising a storage tank for a liquid and a casing, with an opening of the storage tank largely imperviously being sealed with a part of the casing. Moreover, the present invention relates to a storage tank as well as to a casing for an evaporator.

### CROSS REFERENCE TO RELATED CO-PENDING APPLICATIONS

This application is a continuation of International Application No. PCT/DE02/03890 (publication number: WO 03/035157 A1) filed on October 15^{th}, 2002 and entitled VERDUNSTER, INSBESONDERE ATEMLUFTBEFEUCHTER, VORRATSBEHÄLTER SOWIE GEHÄUSE DAFÜR and claims the benefit of the above-mentioned PCT application and the corresponding German National Application Serial Number 101 51 397.6 filed on Oct. 18, 2001 and entitled VERDUNSTER, INSBESONDERE ATEMLUFTBEFEUCHTER, VORRATSBEHÄLTER SOWIE GEHÄUSE DAFÜR the contents of which are expressly incorporated herein by reference.

### FIELD OF THE INVENTION

The present invention relates to an evaporator, a storage tank and a casing therefor used for humidifying air in conjunction with respirators, especially CPAP-apparatus.

### BACKGROUND OF THE INVENTION

Respirators also include so-called CPAP-apparatus, which serve the treatment of apneas during the sleep. To this end, the CPAP (continuous positive airway pressure) therapy was developed, which is described in Chest. Volume No. 110, pages 1077-1088, October 1996 and in Sleep, Volume No. 19, pages 184-188. A CPAP-apparatus generates a positive overpressure up to approximately 30 mbar by means of a compressor or turbine and administers the same, preferably via an humidifier, via a hose and via a nose mask, to the respiratory tract of the patient. This overpressure is to ensure that the upper respiratory tract remains fully opened during the whole night, so that no apneas will occur (DE 198 49 571 A1). An humidifier used in conjunction with said CPAP-apparatus prevents the patient's mucous membranes from desiccating.

A respiratory gas humidifier for CPAP-apparatus is described in DE 199 36 499 A1. The humidifier comprises a refill unit formed of a tub element and a pot part coupled therewith, which can be removed from a mountable casing. The tub element and the pot part are imperviously connected with each other. In conjunction with a partition wall a storage room for a liquid is formed in said pot part, which contains the major part of the water reserve provided for humidifying the respiratory gas. A separate humidifying zone is formed in the tub element disposed underneath the pot part, which merely contains a small portion of the water reserve. The height of the water in the tub element is kept at a predetermined level by a dosing device. In the course of the gradual evaporation of the water located in the tub element water from the liquid storage room is successively or continuously refilled. Via a respiratory gas inlet opening the respiratory gas is blown through the upper portion of the tub element to a respiratory gas outlet opening. The bottom area of the tub element is heated by a heating device. For increasing the thermal transmission, the bottom area of the tub element is made of a material having a high thermal conductivity, e.g. metal.

An humidifier for respirators similar to that described in DE 199 36 499 A1 is described in DE 200 10 553 U1. According to the humidifier disclosed in DE 200 10 553 U1, too, air is passed over the surface of a heatable water reservoir. Instead of a refill unit consisting of a tub element and a pot part, a water tank substantially formed of one part is used. The water tank comprises a filling opening which is closed with a cap during operation.

DE 299 09 611 U1 describes an arrangement for a heatable respiratory humidifier, in which the entire water reserve is heated by a heating element. An air conducting element is provided to allow the air to stroke along closely to the water surface.

DE 298 19 950 U1 describes in connection with a heatable respiratory humidifier a phase control mechanism and a control circuit for controlling the power supplied to a heating element. The heating element heats a water bath.

Documents G-94 09 231.1 and DE 298 17 685 U1 deal with lids for storage vessels for the water reserve of humidifiers for CPAP-apparatus. During operation the lids seal the storage tank in a pressure-proof manner allowing respiratory air to be blown through the upper portion of the storage vessel not filled with water, whereby the lid can slightly be opened for refilling water.

The respiratory humidifier for artificial respiration known from DE 198 08 590 A1 comprises a hose pump as dosing device and an electrically heatable humidifier. The hose pump transports the required amount of water from a commercially available water bag so as to obtain a predetermined relative respiratory gas humidity at a predetermined respiratory gas temperature. The evaporator provides water at a temperature of more than 134° C., which heats the respiratory gas to the predetermined respiratory gas temperature when mixed with the respiratory gas to be humidified. The high heating temperature results from the desire to destroy possibly existing germs in the water.

It is desirable to provide a humidifier comprising a simple storage tank for respirators, a simplified storage tank and a casing for a humidifier.

### SUMMARY OF THE INVENTION

According to an embodiment of the invention an evaporator is provided which comprises a storage tank for a liquid and a casing. An opening of the storage tank largely imperviously closes with a portion of the casing.

According to another embodiment of the invention a storage tank for a liquid with an opening is provided. The opening forms a substantially even rim with a notch.

According to a further embodiment of the invention a casing for an evaporator for accommodating a storage tank is provided. The casing comprises a portion formed such that it substantially imperviously closes with an opening of the storage tank.

In connection with a position at which the edge of the storage tank is less imperviously sealed with a part of the casing it is advantageous that gas can enter the storage tank at this position in a defined manner and that liquid can flow out of the storage tank. By dimensioning this position, moreover, the height of a liquid layer can be defined.

An advantage in the fabrication of a one-piece storage tank resides in that the fabrication is rendered more inexpensive thereby and that the stocking of spare parts is facilitated. Moreover, for cleaning the storage tank, it can be placed in a dishwasher as one piece. Thus, the storage tank need not be disassembled and reassembled, and it is not necessary to place and remove several parts in/from the dishwasher.

An advantage in a gas-proof connection of the storage tank and the casing resides in that the humidifier need not be operated at ambient pressure. The humidifier may rather be regarded as a correspondingly used pressure-proof conducting element between the gas inlet and the gas outlet.

An advantage in the mechanical attachment of the storage tank on the casing by means of a half thread or a bayonet catch resides in that the storage tank can thus easily and quickly be removed from the casing.

An advantage in a thin liquid layer resides in that less power is required for heating the same, because the casing of the storage tank is not co-heated. Moreover, the thickness of the liquid layer is largely independent of the filling level in the storage tank, so that the gas flow through the evaporator is likewise independent of the filling level in the storage tank.

An advantage in the use of a PTC-resistor for the heater resides in that, if the heater's voltage is constant, the heater's temperature fluctuates less strongly than the ambient temperature. On the other hand, the heater's temperature fluctuates, at a constant ambient temperature and a fluctuating heater's voltage, less strongly than it would fluctuate if a temperature-independent resistor for the heater was used. Thus, the use of a PTC-resistor has a temperature-stabilizing effect.

An advantage in the use of a bottom part comprising an electrical terminal resides in that the casing is removed from the bottom part when the storage tank is installed in the casing, so that the power supply to the heater is interrupted and the casing thereby becomes idle.

A gas lock advantageously provides that a considerable gas flow occurs above the liquid layer, i.e. that the gas strokes along closely to the liquid and is effectively enriched with liquid molecules.

### BRIEF DESCRIPTION OF THE DRAWINGS

Preferred embodiments of the invention will hereinafter be explained in more detail with reference to the attached drawings, wherein

FIG. 1 shows a first embodiment of an evaporator according to the invention,

FIG. 2 shows a lateral view of a second embodiment of an evaporator according to the invention, and

FIG. 3 shows a front view thereof.

### DETAILED DESCRIPTION OF THE INVENTION

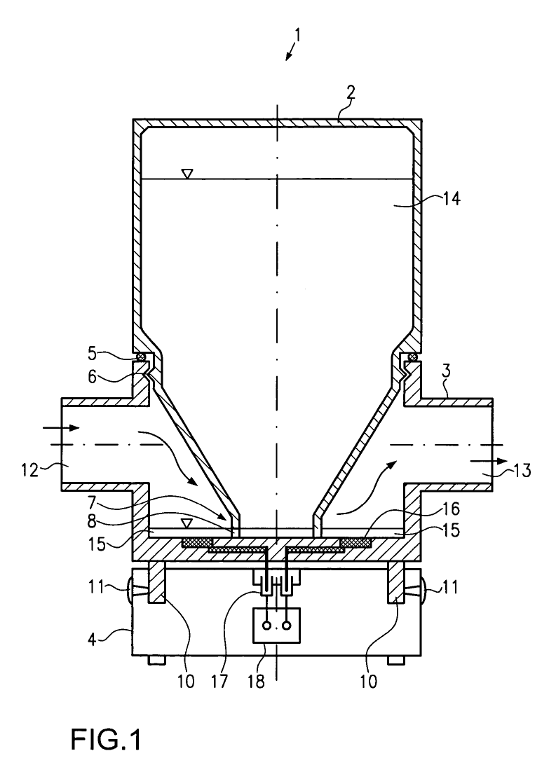

FIG. 1 shows a first embodiment of an evaporator according to the invention, which is formed of a preferably transparent or dull, translucent storage tank 2, a casing 3 and a bottom part 4. An at least translucent storage tank allow an easy, visual filling level control. The storage tank is substantially bottle-shaped. With its opening in a downward direction it is detachably fastened in the casing, preferably by a half thread or a bayonet catch 6.

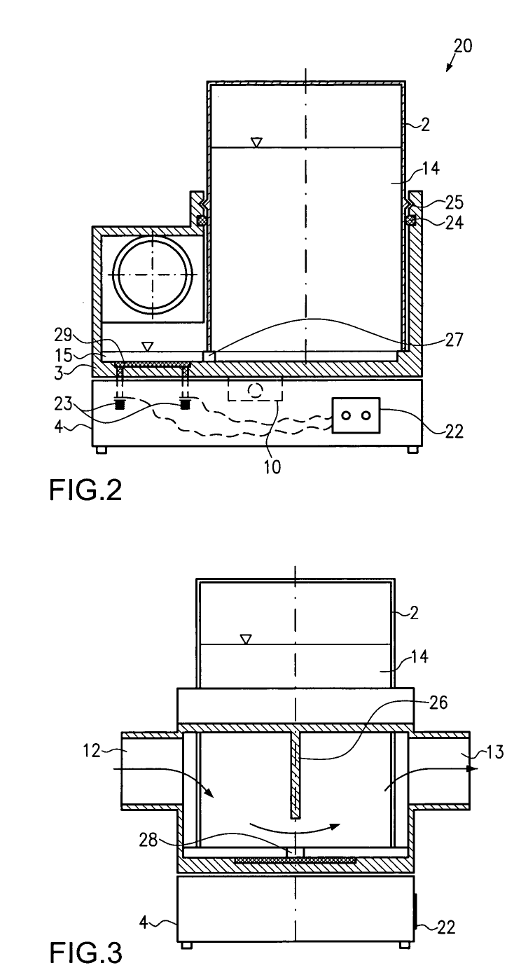

The casing comprises a gas inlet 12 and gas outlet 13. A liquid layer 15 is formed on the bottom of the casing, because liquid 14 flows out of the storage bottle in a defined manner. The dimensions of the casing and the storage bottle are chosen such that the opening of the storage tank substantially imperviously closes with the bottom of the casing. “Substantially imperviously” means that exactly such an amount of liquid flows out of the storage tank 2, that a liquid layer in a desired thickness is formed on the bottom of the casing 3. To this end, a notch 8 may be provided at the opening of the bottle, or a passage 28 (see FIG. 2) in the bottom of the casing 3. With the exception of the position where the notch 8 or the passage 28 are located, the space between the bottom of the casing and the opening of the storage tank is chosen such that, by taking into account the surface tension of the liquid 14, no liquid escapes. Thus, the height of the liquid layer 15 is exclusively determined by the shape and particularly the depth of notch 8, or by the shape and the height of the passage 28. If water is used as liquid, the space between the opening of the storage tank and the bottom of the casing is preferably less than 0.5 mm—with the exception of the position where the notch 8 is located.

As was mentioned above, the storage tank is bottle-shaped. The lower, funnel-shaped part of the storage tank illustrated in FIG. 1 acts like a gas lock 26, because it causes the air to stroke along closely to the liquid layer 15.

The liquid layer 15 is formed next to the opening of the storage tank, as is illustrated in FIG. 1. A preferably annular heating element 16 is disposed underneath the liquid layer. By the evaporation of the liquid from the liquid layer a cold due to the evaporation occurs, so that the liquid layer would cool down and less liquid evaporates per time unit. On the other hand, the vapor pressure of the liquid above the liquid layer can be increased by heating the liquid layer. This purpose is served by the heating element 16. It is preferably made of a PTC-resistor having a strong temperature coefficient. As was mentioned above, this has a temperature-stabilizing effect if the heating element is subject to ambient temperature and voltage fluctuations.

The casing is detachably connected to the bottom part 4 by means of noses 10 and catches 11. According to the preferred embodiment, a terminal 18 to the mains supply (220 or 110 V) is provided at the bottom part. According to this embodiment, in which the heater is operated with supply voltage, a connector is preferably used for electrically connecting the heating element 16 in the casing 3 to the electrical terminal 18 in the bottom part 4. For this purpose, for example, connectors used for water boilers may be used. An embodiment of such a connector comprises plugs connected to the heating element 16 and sockets 17 disposed in the bottom part. The sockets 17 are partially covered by an insulating material, so that they cannot be touched with bare hands—also not by children's hands. According to another embodiment of the connectors, the plugs are designed as one cylindrical plug and one or two hollow-cylindrical plugs arranged coaxially thereto.

For removing and particularly for installing the storage tank in the casing, the casing may be removed from the bottom part by loosening the catches 11. By this, the heating element 16 is separated from the mains supply in an advantageous manner. The storage tank filled with the liquid is held with its opening in an upward direction. Next, the casing is mechanically connected, head first, to the storage tank by means of the half thread or bayonet catch 6. Next, the casing with the storage tank is turned upside down, so that the casing is at the bottom side and the storage tank with the opening faces downwardly, as is illustrated in FIG. 1. When turning the same, only a small amount of liquid escapes from the storage tank into the casing, namely because the opening of the storage tank is largely imperviously closed with the bottom of the casing. Next, liquid slowly flows from the storage tank into the casing, so that a liquid layer having a predetermined thickness is formed.

Now, the casing is inserted into the bottom part, so that the catches 11 are locked into place.

The seal 5 is provided for allowing the storage tank 2 and the casing 3 to form a largely gas-proof unit. In this way, the escape of gas from the casing into the environment can largely be prevented, even if a slight overpressure of 10 to 200 mbar compared to the ambient pressure prevails in the casing. The diameter of the opening of the storage tank should not be smaller than approximately 1/3 of the bulge of the storage tank, which is the part of the storage tank positioned above seal 5 in FIG. 1. These dimensions allow the cleaning of the storage tank in a dishwasher.

FIGS. 2 and 3 show an additional embodiment of an evaporator according to the invention. This evaporator is likewise formed of a preferably transparent storage tank 2, a casing 3 and a bottom part 4. Again, the storage tank 2 is mechanically connected with the casing preferably by means of a half thread or a bayonet catch. In this embodiment, too, a seal 24 is provided so as to connect the storage tank and the casing with each other in a largely pressure-proof manner.

According to this embodiment the storage tank is cup-shaped. The cleaning of the storage tank becomes very easy due to the large opening. For largely sealing the storage tank with the casing a web 27 is provided. For allowing the liquid to flow out in a defined manner and for forming a liquid layer 15 of a certain thickness, passage 28 is provided. If water is used as liquid, the space between the web and the storage tank should, for the above-mentioned reasons, be smaller than 0.5 mm so as to render the web and the storage tank substantially liquid-proof. As is shown in FIG. 2, the liquid layer 15 is formed next to the opening of the storage tank, namely underneath the gas inlet and the gas outlet 12, 13. A heating element 29 is disposed underneath the liquid layer. According to this embodiment, a low-voltage heating element operating at a supply voltage between 5 and 24 V is used. An advantage in this embodiment resides in that it can also be operated in the supply system of a car or a truck. A transformer may be provided for a mains supply operation (110 or 220 V). A gas lock 26 is provided between the gas inlet 12 and the gas outlet 13 to allow the gas to stroke along closely to the liquid surface.

The storage tank is mounted in the casing in a fashion similar to that described above in connection with FIG. 1. At first, the casing is removed from the bottom part, inversely placed upon the upwardly open storage tank and screwed tightly with the same. Again, the storage tank and the casing are together turned upside down and fastened to the bottom part.

The electrical connection between the electrical terminal 22 and the heater 29 can take place precisely as illustrated in FIG. 1. Since, however, a low-voltage is used, the contacts in the bottom part need not be insulated against contact. Therefore, contact pins 23 for contacting the heating element 29 may be used.

According to the embodiment illustrated in FIGS. 2 and 3, however, also a heater constructed for a supply voltage (110 or 230 V) can be employed. For electrically connecting the casing and the bottom part the above-described sockets protected against contact have to be used in this case. According to the embodiment illustrated in FIG. 1 a low-voltage heater having an operational voltage between 5 and 24 V may analogously be employed. In this case, also contact spring pins 23 may be used for contacting the heater.

Finally, according to the embodiment illustrated in FIGS. 2 and 3, one or more notches may be provided in the storage tank 2 instead of the web 27 with a passage 28. According to the embodiment shown in FIG. 1 a web 27 may be provided to seal the storage tank with the bottom of the casing. Instead of the notch 8 in the storage tank 2, said web may also have a passage 28.

In another embodiment a passage may also be provided together with a notch downstream of the opening of the storage tank.

The gas inlet 12 is preferably connected with respirators, e.g. a CPAP-, BiPAP- or a multilevel apparatus. The gas outlet, on the other hand, is connected to the patient's mask. The embodiments of this evaporator according to the invention may also be integrated in a CPAP-, BiPAP- or a multilevel apparatus. To this end, the turbine and the control mechanism of the CPAP-apparatus are preferably disposed on the bottom part 4. According to the latter embodiment the gas inlet is designed such that, when the casing 3 is affixed on the bottom part 4, the gas inlet of the humidifier is positioned opposite a gas outlet of the turbine of the CPAP-apparatus and is sealed with the same in a largely air-tight manner.

According to other embodiments the heating elements 16 and 29 may be arranged in the bottom part 4. In these embodiments, the portion of the casing 3 located underneath the thin liquid layer 15 is constructed to have a good thermal conductivity. This may take place by constructing this portion of the casing particularly thin. Alternatively or additionally, this portion may also be made of a metal with a good thermal conductivity, e.g. metal. An advantage in view of the latter embodiments is that electric contacts between the bottom part 4 and the casing 3 become superfluous. It may prove to be a drawback that, after the casing 3 is removed, the heating element 16 or 29 is freely accessible, which may entail burns upon contact. It is self-evident, that the gap between the bottom part 4 and the casing 3 illustrated in FIG. 1 to 3 for reasons of clarity is dimensioned as narrow as possible according to these embodiments.

In the foregoing, the invention was explained in more detail by means of preferred embodiments. For a person skilled in the art it is obvious, however, that different modifications may be made, without deviating from the spirit of the invention. Therefore, the scope of protection is defined by the following claims and the equivalents thereof.

### List of Reference Numerals

1 evaporator

2 storage tank

3 casing

4 bottom part

5 seal

6 half thread or bayonet catch

7 opening

8 notch

10 spike

11 catch

12 gas inlet

13 gas outlet

14 liquid

15 liquid layer

16 heating element

17 sockets

18 electrical terminal

20 evaporator

22 electrical terminal

23 contact spring pins

24 seal

25 half thread or bayonet catch

26 gas lock

27 web

28 passage

29 heating element

What is claimed is:

1. Evaporator, especially a respiratory humidifier, comprising:
a storage tank for a liquid; and
a casing; an opening of the storage tank largely imperviously closing with a portion of the casing.

2. Evaporator according to claim 1, wherein there is a position at which the edge of the opening of the storage tank is spaced away from the portion of the casing farther than usual.

3. Evaporator according to claim 1, wherein the storage tank is made of one piece.

4. Evaporator according to claim 1, wherein the casing comprises a gas inlet and a gas outlet, that a seal is arranged between the casing and the storage tank so as to produce a gas-proof connection between the casing and the storage tank, and that a half thread is provided to fasten the storage tank on the casing.

5. Evaporator according to claim 1, wherein the casing comprises a gas inlet and a gas outlet, that a seal is arranged between the casing and the storage tank so as to produce a gas-proof connection between the casing and the storage tank, and that a bayonet catch is provided to fasten the storage tank on the casing.

6. Evaporator according to claim 1, wherein the casing is constructed such that, when the storage tank is installed and at least partially filled with liquid, a thin liquid layer is formed in the casing next to the opening of the storage tank, above which gas can be passed along.

7. Evaporator according to claim 5, wherein a heater is disposed underneath the position provided for the thin liquid layer.

8. Evaporator according to claim 7, wherein the heater is made of a PTC-resistor.

9. Evaporator according to claim 1, wherein the evaporator further comprises a bottom part having an electrical terminal detachably connected to the casing by a spike and a catch.

10. Evaporator according to claim 6, wherein the evaporator further comprises a bottom part having an electrical terminal detachably connected to the casing by a spike and a catch, the electric contact between the heater and the electrical terminal being produced via contact surfaces on the casing and contact spring pins in the bottom part.

11. Evaporator according to claims 6, wherein the evaporator further comprises a bottom part having an electrical terminal detachably connected to the casing by a spike and a catch, the electric contact between the heater and the electrical terminal being produced via plugs mechanically connected to the upper part and sockets arranged in the bottom part in a hidden manner.

12. Evaporator according to claim 4, wherein a gas lock is located between the gas inlet and the gas outlet.

13. Storage tank for a liquid with an opening, wherein the opening forms a substantially even rim with a notch.

14. Casing for an evaporator for accommodating a storage tank, wherein the casing comprises a portion formed such that it substantially imperviously closes with an opening of the storage tank.
